(19) United States
(12) Patent Application Publication
(10) Pub. No.: US 2015/0150983 A1
(43) Pub. Date: Jun. 4, 2015

(54) Intra-articular Formulations and Methods for Treatment of Osteoarthritis

(71) Applicant: DePuy Mitek, LLC, Raynham, MA (US)

(72) Inventors: Benjamin A. Byers, North Easton, MA (US); William R. Parrish, Hudson, MA (US); Brooks J. Story, Franklin, MA (US); Jeffrey Geesin, Doylestown, PA (US); Uri Herzberg, Raynham, MA (US); Scott A. Wadsworth, New Hope, PA (US)

(21) Appl. No.: 14/554,689

(22) Filed: Nov. 26, 2014

Related U.S. Application Data
(60) Provisional application No. 61/910,562, filed on Dec. 2, 2013.

(51) Int. Cl. A61K 47/26; A61K 9/00; A61K 47/10; A61K 38/18

(52) U.S. Cl. CPC A61K 47/26; A61K 38/1875; A61K 9/0019; A61K 47/10

(57) ABSTRACT

Intra-articular formulations and methods are provided for treating joint conditions, such as osteoarthritis. The formulations include an effective amount of recombinant human growth differentiation factor-5 (rhGDF-5) and an acidic vehicle, where the formulation is an intra-articular injection formulation. The formulations described herein can be used for treating joint conditions (e.g., osteoarthritis) via intra-articular injection in a subject in need thereof.

### RELATED APPLICATIONS

This application claims priority to, and the benefit of, U.S. Provisional Application No. 61/910,562, filed on Dec. 2, 2013, which is incorporated herein by reference in its entirety.

### FIELD

The present invention relates generally to formulations and methods for treating joint conditions.

### BACKGROUND

Osteoarthritis (“OA”), the most common form of arthritis, is a type of arthritis that is characterized by degenerative (gradual deterioration of joint) or abnormal changes in bone, cartilage, and synovium of the joints. OA is a leading cause of disability in the USA, Europe and Japan. The estimated total prevalence of radiographic OA in these countries ranges from 36% to 48% of the population; and this number is expected to increase annually due to the growing proportion of older age groups and an increase in risk factors for OA, primarily obesity and an inactive life style.

Treatment of OA generally involves a combination of exercise, physical therapy, lifestyle modification, and analgesics. Acetaminophen is typically the first line treatment for OA. For mild to moderate symptoms, effectiveness is similar to non-steroidal anti-inflammatory drugs (“NSAIDs”), such as ibuprofen. For more severe symptoms NSAIDs may be more effective. However, while more effective, NSAIDs in severe cases are associated with greater side effects such as gastrointestinal bleeding and renal complications. Another class of NSAIDs, COX-2 selective inhibitors (such as Celecoxib), is equally effective to NSAIDs but no safer in terms of side effects. There are several NSAIDs available for topical use, including diclofenac. Typically, they have less systemic side-effects than oral administration and at least some therapeutic effect. While opioid analgesics, such as morphine and fentanyl, improve pain this benefit is outweighed by frequent adverse events and thus they are not routinely used. Intra-articular steroid injections are also used in the treatment of OA, and they are very effective at providing pain relief, especially in patients exhibiting inflammatory elements of OA. However, the duration of the pain relief is limited to 4-6 weeks and there are adverse effects that may include collateral cartilage damage. If pain becomes debilitating, joint replacement surgery may be used to improve mobility and quality of life. There is no proven treatment to slow or reverse the disease.

Therefore, there is still a large unmet need for improved methods and compositions for treating joint conditions, particularly methods and compositions that can provide effective long-term disease-modifying and symptomatic treatment.

### SUMMARY

Provided herein is an injectable formulation that includes an effective amount of recombinant human growth differentiation factor-5 (rhGDF-5) and an acidic vehicle, where the formulation is an intra-articular injection formulation.

In some embodiments, the acidic vehicle contains trehalose, sucrose, raffinose, glucose, mannitol, sorbitol, erythritol or any combination thereof. Typically, the acidic vehicle contains trehalose.

In some embodiments, the trehalose is about 0.1% to 50% by weight of the formulation. Typically, the trehalose is about 5% by weight of the formulation.

In some embodiments, the formulation has a pH of from about 2.5 to about 5.0. Typically, the formulation has a pH of about 3.

In some embodiments, the effective amount of rhGDF-5 is about 1.2 mg to about 30 mg.

In some embodiments, the formulation includes a diluent selected from the group consisting of water, saline, and a buffer. Typically, the buffer is a glycine buffer.

Also provided herein is a method of treating a joint condition in a subject in need thereof by administering to the subject via an intra-articular injection an acidic composition including an effective amount of rhGDF-5.

In some embodiments, the method includes a single injection in a treatment period.

In some embodiments, the method includes two bi-weekly injections in a treatment period.

In some embodiments, the method includes three bi-weekly injections in a treatment period.

In some embodiments, the acidic composition contains trehalose. Typically, the acidic composition contains glycine buffered trehalose.

In some embodiments, the acidic composition has a pH of from about 2.5 to about 5.0. Typically, the acidic composition has a pH of about 3.

In some embodiments, the effective amount of rhGDF-5 is about 1.2 mg to about 30 mg.

In some embodiments, the joint condition is osteoarthritis.

In some embodiments, the effective amount of rhGDF-5 stimulates cartilage regeneration.

In some embodiments, the effective amount of rhGDF-5 reduces the width of at least one bone lesion.

Further provided herein is a kit encompassing a container that includes a solution having a pH in the range of about 2.5 to about 5.0 containing an acidic vehicle and an effective amount of rhGDF-5; and a syringe for injection of the solution.

In some embodiments, the effective amount of rhGDF-5 in the solution is about 1.2 mg to about 30 mg.

In some embodiments, the acidic vehicle includes trehalose.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims. The citation of any reference herein should not be deemed as an admission that such reference is available as prior art to the instant invention.

### BRIEF DESCRIPTION OF THE DRAWINGS

The appended drawings have been included herein so that the above-recited features, advantages and objects will become clear and can be understood in detail. These drawings form a part of the specification. It is to be noted, however, that the appended drawings illustrate exemplary embodiments and should not be considered to limit the scope.

FIG. 1 is a bar graph showing that rhGDF-5 has demonstrated significant dose-dependent cartilage protection by measuring the significant tibial cartilage degeneration width (micron), when compared with vehicle controls (“V”);

FIG. 2 is a table showing occurrence of ectopic bone, inflammation index, medial collateral ligament (MCL) thickness and osteophyte size in each group of animal with different treatment regimens;

FIG. 3 is a bar graph showing the size of osteophytes that form in the treated knee as a result of the MMT surgery, demonstrating significant effects of OP-1 on osteophyte formation.

FIG. 4 is a bar graph that has demonstrated dose-dependent efficacy of rhGDF-5 in treating osteoarthritis compared with vehicle controls;

FIG. 5 is a bar graph that demonstrates the dose-dependent efficacy of rhGDF-5 in treating osteoarthritis compared with vehicle controls; and

FIG. 6 is a series of histological staining results showing that treatment of rhGDF-5 in an osteoarthritis model successfully reduced the width of tibial lesion.

### DETAILED DESCRIPTION

Certain exemplary embodiments will now be described to provide an overall understanding of the formulations of rhGDF-5 and use of such formulations for treating joint conditions disclosed herein. Those of ordinary skill in the art will understand that the formulations and methods specifically described herein are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e., to at least one) of the grammatical object of the article. By way of example, “an acidic vehicle” means one acidic vehicle or more than one acidic vehicle.

The terms “comprise,” “include,” and “have,” and the derivatives thereof, are used herein interchangeably as comprehensive, open-ended terms. For example, use of “comprising,” “including” or “having” means that whatever element is comprised, had, or included, is not the only element encompassed by the subject of the clause that contains the verb.

All numerical designations (e.g., pH, dose, and concentration, including ranges) are approximations which are varied (+) or (−) by increments of 1.0 or 0.1, as appropriate. It is to be understood, although not always explicitly stated that all numerical designations are preceded by the term “about.” It also is to be understood, although not always explicitly stated, that the reagents described herein are merely exemplary and that equivalents of such are known in the art.

A “therapeutically effective amount” or “effective amount” is that amount of an agent to achieve a pharmacological effect. The term “therapeutically effective amount” includes, for example, a prophylactically effective amount. An “effective amount” is an amount sufficient to achieve a desired pharmacologic effect or therapeutic improvement without undue adverse side effects. For example, an effective amount refers to an amount that increases operativity, or increases weight bearing load, or decreases pain, or increases growth in the bone and cartilage of one or more joints, or reduces joint distortion, pain, swelling, or stiffness. The effective amount of an agent will be selected by those skilled in the art depending on the particular patient and the disease level. It is understood that “an effective amount” or “a therapeutically effective amount” can vary from subject to subject, due to variation in metabolism of therapeutic agents such as s and/or prokinetic agents, age, weight, general condition of the subject, the condition being treated, the severity of the condition being treated, and the judgment of the prescribing physician.

“Treat” or “treatment” refers to any treatment of a disorder or disease associated with bone or cartilage disorder, such as preventing the disorder or disease from occurring in a subject, which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping at least one symptom of the disease or disorder. Thus, as used herein, the term “treat” is used synonymously with the term “prevent.”

By “co-administered” is meant simultaneous administration in the same formulation or in two different formulations that are combined into one formulation for administration.

The term “subject” as used herein refers to an animal, in some embodiments, a mammal and in some embodiments, a human, who can benefit from the formulations, compositions and methods described herein. There is no limitation on the type of animal that could benefit from the present methods. A subject regardless of whether a human or non-human animal may be referred to as an individual, subject, animal, host or recipient. The methods described herein have applications in human medicine, veterinary medicine as well as in general, domestic or wild animal husbandry. In one embodiment, the candidate subject is a mammal such as a human; laboratory test animal, such as a mouse, rat, rabbit, guinea pig, hamster; avian species, such as a poultry bird; or veterinary medical animal, such as dog, cat, horse, cow, sheep, etc.

In general, described herein are formulations and methods for treating joint conditions, such as osteoarthritis and/or the pain associated therewith. The formulations and methods utilize an effective amount of recombinant human growth differentiation factor-5 (rhGDF-5) in an acidic vehicle.

Formulations

Due to the localized nature of the joint conditions (such as osteoarthritis and/or the pain associated therewith), intra-articular (IA) drug injection is an attractive treatment approach for OA. However, the various glucocorticoid and hyaluronic acid (HA) IA formulations, which are currently available on the market for OA treatment, provide only short-term pain relief or/and often do not provide adequate symptomatic treatment, let alone provide adequate disease-modifying treatment. Another obvious disadvantage of currently available IA injections is the discomfort and pain they may cause and the possible risk of infection introduced by the injections. The available oral drugs (such as the non-selective NSAIDs and selective cyclooxygenase 2 inhibitors) for symptomatic treatment also have shortcomings, most notably side effects, including gastrointestinal complications and cardiovascular side effects and risks. Other bone growth factors (such as FGF-18 and BMP-7) have been previously used for enhancing bone fusion and cartilage repair. However, these factors were observed to induce formation of ectopic bone and/or cartilage in the synovial tissue.

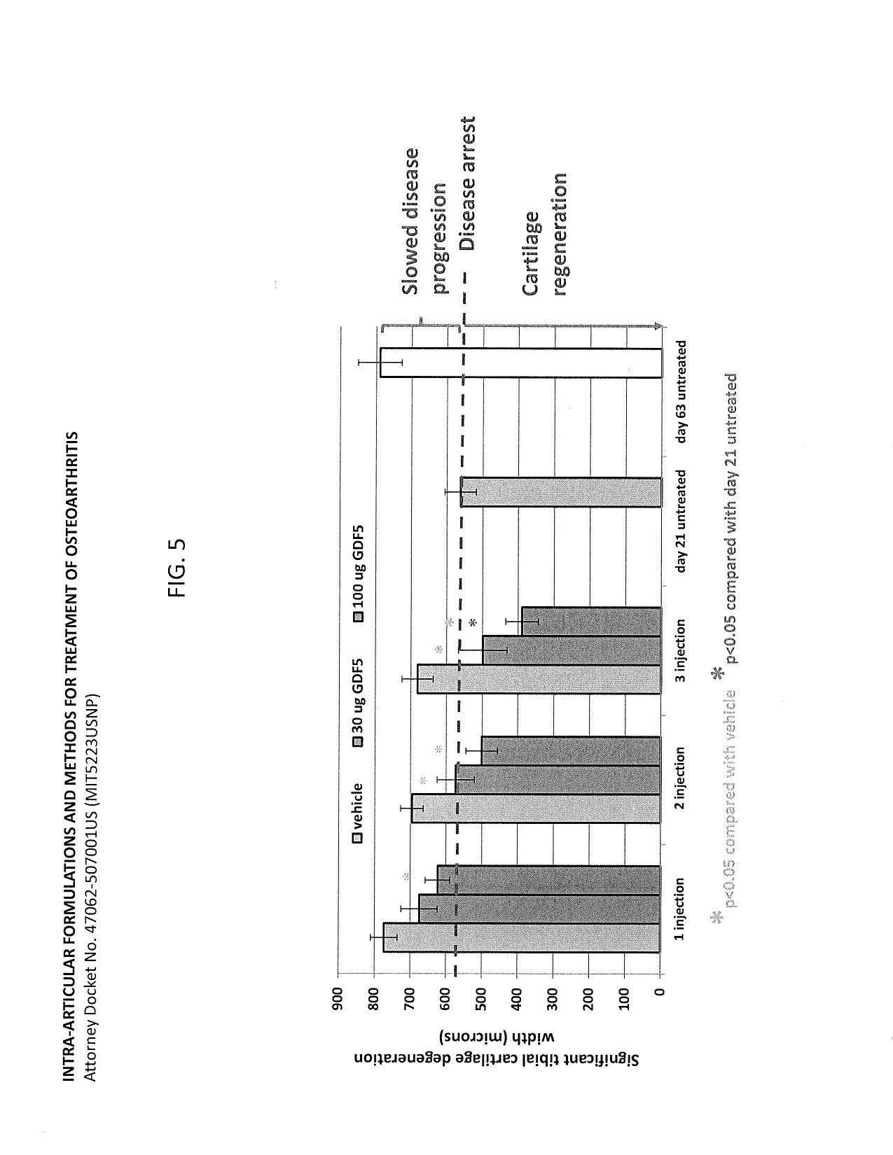

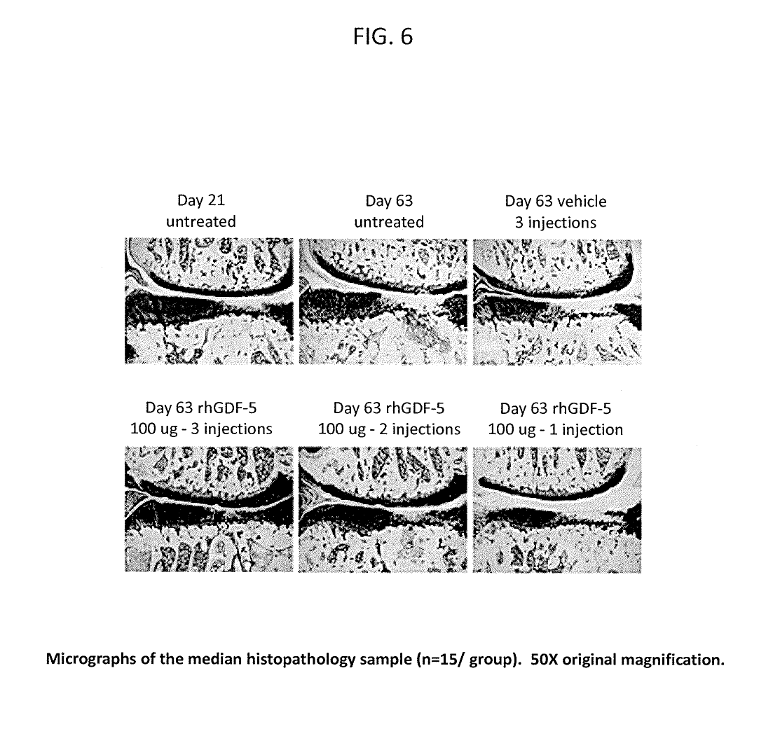

The formulations described herein provide a superior and unexpected solution to solve these problems. First, the formulations provide excellent efficacy in treating a joint condition. As clearly demonstrated in FIGS. 5 and 6 and Example 3, as low as a single injection can successfully reduce the width of significant tibial lesions when compared to the control groups. In fact, the formulations described herein can stimulate cartilage regeneration (see Example 3), thus providing not only symptomatic relief, but also disease-modifying treatment (e.g., cure of the disease). This great efficacy of the present formulations thus provides a variety of benefits to the patients. For example, compared to other known IA formulations, the instant formulation requires a lower frequency of injections and/or much shorter treatment period, and therefore causes less discomfort and pain and reduces the risk of infection that may be introduced by each injection. In addition, higher efficacy, lower frequency of injections and/or shorter treatment period can also significantly reduce the cost or spending for treating OA.

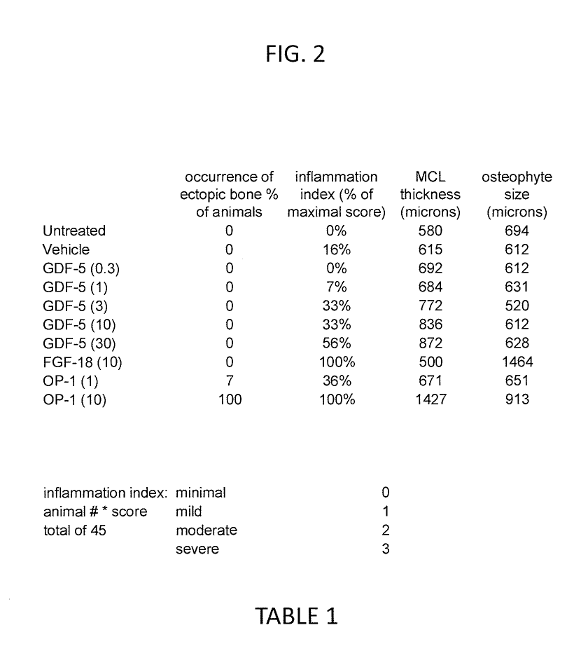

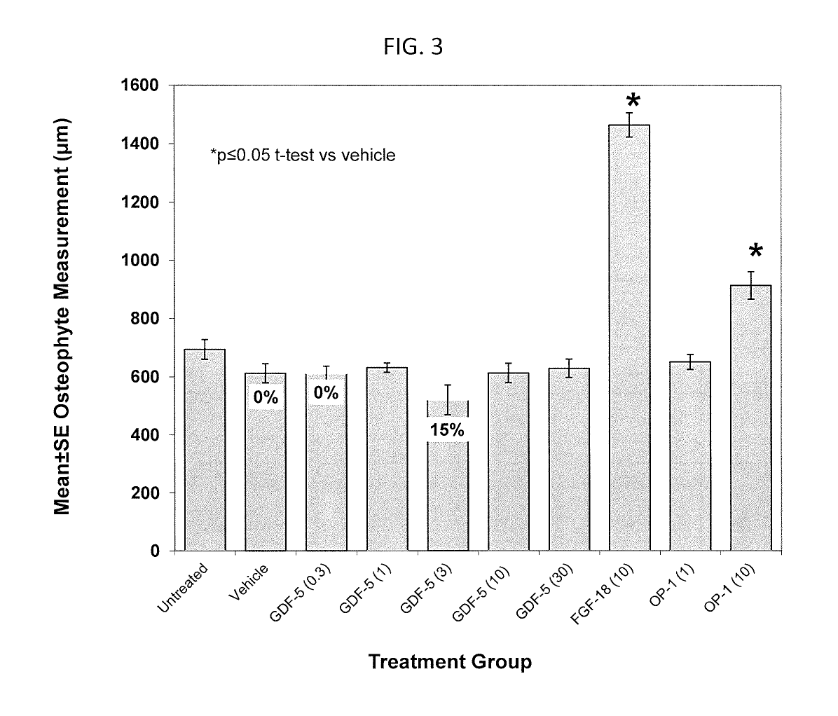

Second, the formulations are not believed to cause any side effects, such as formation of ectopic bone and/or cartilage in the synovial tissue. As shown in FIG. 2 (Table 1), FIG. 3 and Example 1, treatment by other bone growth factors (such as FGF-18 and BMP-7) caused formation of ectopic bone and/or cartilage in the synovium, despite showing a certain level of cartilage preservation. This detrimental side effect makes it impossible, or at least much less ideal, for these other bone growth factors as therapeutic candidates for treating joint conditions. In contrast, rhGDF-5 and the formulations described herein present an excellent therapeutic approach for treating a joint condition without having such side effect.

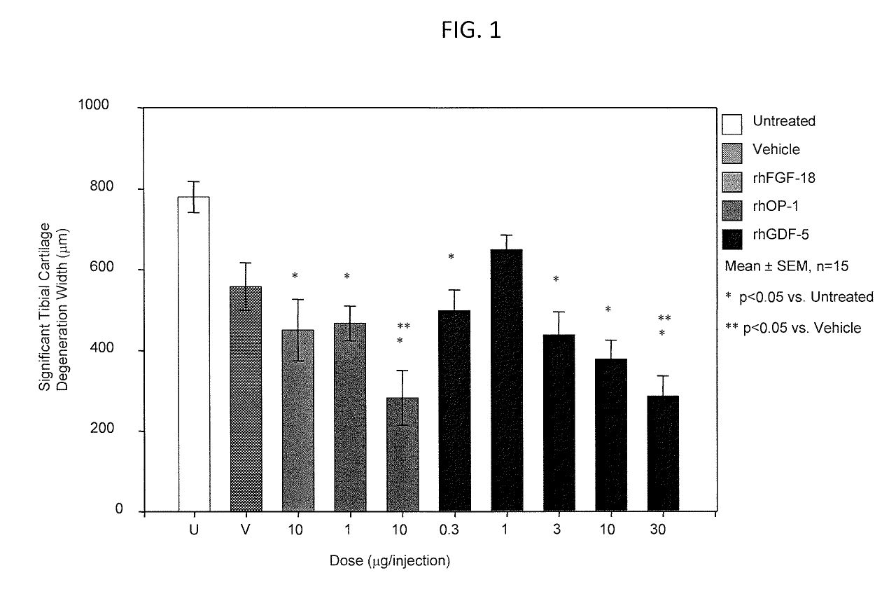

Third, the acidic vehicle used in the formulation also provides unexpected and superior effects. It is well recognized in the art that the pH of the available IA formulations is either close to the pH of synovium fluid (i.e., pH 7.4) or slightly lower, but not below pH ˜5.5, to allow for optimum stability of the active ingredient, while minimizing possible side effects of non-physiological pH values, such as pain or tissue damage at the injection site. However, the use of an acid vehicle in the present formulations surprisingly produced unexpected benefits. In fact, the acidic vehicle alone showed an unexpected effect on reducing cartilage erosion (see FIG. 1). Further, the acidic vehicle did not cause any side effects (such as pain, inflammation or tissue damage at the injection site) that one would have expected for an acidic IA formulation based on the current knowledge. The acidic vehicle provides the additional benefit that it functions as an excellent solvent for rhGDF-5, which would otherwise precipitate and cannot be used for IA formulations. The acidic vehicle also provides an excellent preserved solution against the contaminating action of microorganisms such as bacteria and fungi. Such a preserved solution not only provides an excellent sterile environment, but also makes it easier to produce a commercial kit with longer shelf-life.

Accordingly, provided herein is an injectable formulation that contains an effective amount of rhGDF-5 and an acidic vehicle, where the formulation is an intra-articular injection formulation. Typically, the formulation is in a liquid form.

rhGDF-5 can be obtained by any recombinant technology known in the art. Typically, prokaryotic expression vectors such as bacterial plasmids are used for producing rhGDF-5. Expression of GDF-5-related proteins using recombinant DNA techniques has been described in Hötten, U.S. Pat. No. 6,764,994; Makishima, U.S. Pat. No. 7,235,527; Ehringer, U.S. Pat. No. 8,187,837, each of which is incorporated herein by reference. rhGDF-5 can be formulated via a lyophyilization process as described in U.S. Pat. No. 8,455,436, which is incorporated herein by reference in its entirety.

The effective amount of rhGDF-5 present in the formulation can vary. In some embodiments, the effective amount of rhGDF-5 is about 1.2 mg to about 30 mg in the formulation per injection. For example, the effective amount of rhGDF-5 is about 1.2 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg or 30 mg. In some embodiments, the effective amount of rhGDF-5 is about 1.8 mg to about 6 mg in the formulation per injection.

The concentration of rhGDF-5 present in the formulation can also vary, but in an exemplary embodiment rhGDF-5 is provided at a therapeutically effective amount. In some embodiments, the final concentration of rhGDF-5 in the formulation is about 0.6 mg/ml to about 3.0 mg/ml per injection. For example, the final concentration of rhGDF-5 in the formulation is about 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3 mg/ml or any range derivable therein. Lower concentrations may be used if more frequent injections are used.

In some embodiments, the volume of the formulation is about 2.0 ml to about 10.0 ml per injection. For example, the volume of the formulation is about 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml or 10 ml.

It is desirable that the formulations described herein have a pH of about 2.5 to about 5.0. For example, the formulation has a pH value of about 2.5 to about 4.0, about 2.5 to about 3.5 or about 2.5 to about 3.0. For example, the formulation has a pH value of about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5. Typically, the formulation has a pH value of about 3.0.

Accordingly, in some embodiments, the formulations of the invention may further comprise one or more buffering agents or combinations thereof that are used to adjust and/or maintain the formulations into the desired pH range. Adjustment of pH or buffering agents that are suitable for use in the formulations of the invention include, but are not limited to, glycine, citric acid, sodium citrate, sodium phosphate (dibasic, heptahydrate form), and boric acid or equivalent conventional buffers, or combinations thereof. Typically, the formulation is glycine buffered. The appropriate amounts of buffers and buffering agents, or combinations thereof, that are to be used in the formulations of the invention are readily determined by those of ordinary skill without undue experimentation, particularly in view of the guidance contained herein and in standard formularies such as the United States Pharmacopoeia, Remington: The Science and Practice of Pharmacy, and the like, the disclosures of which are incorporated herein by reference in their entireties.

In some embodiments, the acidic vehicle of the formulation contains trehalose, sucrose, raffinose, glucose, mannitol, sorbitol, erythritol or any combination thereof. Typically, the acidic vehicle contains trahalose.

In some embodiments, the trehalose is about 0.1% to about 50% by weight of the formulation. For example, the trehalose is about 0.10%, 0.50%, 0.10%, 0.50%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50% or 50.00% by weight of the formulation. Typically, the trehalose is about 5.0% by weight of the formulation.

In some embodiments, the formulation contains at least one stabilizer. Stabilizers can be sugars or derivatives, such as saccharides, disaccharides, modified saccharides, sugar alcohols, or polysaccharides. In an exemplary embodiment, the stabilizer can be tocopherol, tocopherol derivatives, glucose, mannitol, sucrose, sorbitol, erythritol and/or trehalose.

Common stabilizers or stabilizing excipients used in the pharmaceutical industry are saccharides, disaccharides, modified saccharides, sugar alcohols and polysaccharides. Some of these molecules are commonly used as excipients because they can also serve as a sweetening agent when the drug is in a tablet form for oral delivery. According to U.S. Pat. No. 7,351,798, hyaluronic acid and glycosaminoglycans can also be excipients.

Trehalose (α-D-glucopyranosyl α-D-glucopyranoside), a disaccharide known for its antioxidant properties, has been known as a non-reducing saccharide consisting of glucoses. As is described in Advances in Carbohydrate Chemistry, Vol. 18, pp. 201-225 (1963), published by Academic Press, USA, and Applied and Environmental Microbiology, Vol. 56, pp. 3,213-3,215 (1990), trehalose widely exists in microorganisms, mushrooms, insects, etc., though the content is relatively low. Trehalose is a non-reducing saccharide, so that it neither reacts with substances containing amino groups such as amino acids and proteins, induces the amino-carbonyl reaction, nor deteriorates amino acid-containing substances. Thus, trehalose can be used without a fear of causing an unsatisfactory browning and deterioration.

Trehalose can also inhibit the inflammatory cascade, thereby suppressing cytokine production. (Minutoli, et al, SHOCK, Vol. 27, No. 1, pp. 91-96, 2007; and Chen Q, Haddad GG: Role of trehalose phosphate synthase and trehalose during hypoxia: from flies to mammals. J Exp Biol 207:3125-3129, 2004.) Trehalose is a unique sugar capable of protecting biomolecules against environmental stress and may inhibit the inflammatory cascade that in turn causes oxidative damage and cytokines production. Trehalose has also been shown to preserve cell viability, during exposure to a range of environmental stress, such as heat shock, dehydration and hypoxia.

Trehalose is also a common food additive because it is a strong antioxidant and sweetener, and it is often used as a stabilizing agent in pharmaceutical preparations. Trehalose, like sucrose, is a non-reducing disaccharide (two glucose molecules linked by the anomeric carbon) that can act as an effective lyoprotectant for the freeze drying of proteins and other biomolecules. During the freeze drying process, proteins can denature as water is removed unless a substitute molecule is available to support the structure of the protein. Trehalose fills the void left by exiting water and prevents this denaturation. When used at concentrations as low as 2% it can effectively protect proteins and other biomolecules.

Useful forms of trehalose can include trehalose dihydrate (TD) which is crystalline, amorphous trehalose (AT) which is a vitreous form, and the anhydrous forms of trehalose, anhydrous amorphous trehalose (AAT) and anhydrous crystalline trehalose (ACT). Powdered anhydrous trehalose may contain AAT and/or ACT. The term “trehalose,” as used herein, refers to any physical form of trehalose including anhydrous, partially hydrated, fully hydrated and mixtures and solutions thereof. The manufacture and use of anhydrous trehalose from TD can be found in International Publication No.: PCT/GB97/00367, the disclosure of which is incorporated into this specification by reference.

Trehalose can be present in liquid, solid, lyophilized or crystalline forms. When present in liquid form, trehalose can be in a buffered solution. Solvents that can be used to solubilize trehalose can include, but are not limited to, water, saline or other salt solutions, buffer solutions such as phosphate buffered saline, histidine, lactate, succinate, glycine, and glutamate, dextrose, glycerol, as well as combinations thereof. Particularly, trehalose can be present in the rhGDF-5 formulation as a solution.

The concentration of the at least one stabilizer present in the formulation can vary, but in an exemplary embodiment at least one excipient is provided at a pharmaceutically effective amount. In an exemplary embodiment, the at least one stabilizer has a concentration of at least about 1 mg/ml, at least about 5 mg/ml, at least about 50 mg/ml or at least about 100 mg/ml, and in some embodiments the concentration can be at least about 200 mg/ml. Suitable concentrations of at least one stabilizer can include about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/mg, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, 41 mg/ml, 42 mg/ml, 43 mg/ml, 44 mg/ml, 45 mg/ml, 46 mg/ml, 47 mg/ml, 48 mg/ml, 49 mg/ml, 50 mg/ml, 51 mg/ml, 52 mg/ml, 53 mg/ml, 54 mg/ml, 55 mg/ml, 56 mg/ml, 57 mg/ml, 58 mg/ml, 59 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 160 mg/ml, 170 mg/ml, 180 mg/ml, 190 mg/ml, 200 mg/ml, 300 mg/ml, 400 mg/ml, 500 mg/ml, 600 mg/ml or more or any range derivable therein. Stabilizers can also be in a concentration in a range of about 0.1-60% by weight; about 0.1-50% by weight, about 0.1-45% by weight; or about 0.1-20% by weight. Other suitable concentrations of at least one excipient can include about 0.1%, 0.5%, 1%, 2.5%, 5%, 6%, 7%, 8%, 9% 10%, 11%, 12%, 13%, 14%, 15%, 16%, 17%, 18%, 19%, 20%, 25%, 30%, 35%, 40%, 45%, 50%, 55%, or about 60% by weight.

In some embodiments, the formulations described herein may further include one or more pharmaceutically acceptable carriers. Exemplary pharmaceutically acceptable carries include, but are not limited to, antimicrobial agents, isotonic agents, buffers, antioxidants, local anesthetics, suspending and dispersing agents, emulsifying agents, sequestering agents, chelating agents and any combinations thereof. Frequently used “carriers” or “auxiliaries” include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols, and dimethyl sulfoxide. Intravenous vehicles include fluid and nutrient replenishers. Preservatives include antimicrobial agents, antioxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co.: 1405-1412, 1461-1487, 1975 and The National Formulary XIV., 14th ed. Washington: American Pharmaceutical Association, 1975 the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The rhGDF-5 in an acidic vehicle and the at least one additional component, such as an excipient, a pharmaceutically acceptable carrier or a stabilizer, can be configured to be combined and administered intra-articularly independent of a surgical procedure, or as part of a surgical procedure involving an articulating joint, either immediately before, during or immediately after the surgical procedure. Alternatively, the rhGDF-5 in an acidic vehicle and at least one additional component can be previously combined and present as a combination formulation at the time of the surgical procedure. The other components, when combined, can form a resulting composition or mixture having each component present in the composition at various amounts. The amount of each component in the composition can vary.

The formulations to be used for in vivo administration must be sterile. This is readily accomplished by filtration through sterile filtration membranes. Use of such membrane filters can eliminate the need for preservatives in the various liquid formulations of the present invention. However, certain liquid compositions of the invention may further comprise one or more preservatives and/or one or more stabilizers. Preservatives that are suitable for use in the compositions of the invention include, but are not limited to, edetic acid and their alkali salts such as disodium EDTA (also referred to as “disodium edetate” or “the disodium salt of edetic acid”) and calcium EDTA (also referred to as “calcium edetate”), benzyl alcohol, methylparaben, propylparaben, butylparaben, chlorobutanol, phenylethyl alcohol, benzalkonium chloride, thimerosal, propylene glycol, sorbic acid, and benzoic acid derivatives. The preservatives should be used at a concentration of from about 0.001% to about 0.5% (w/v) in the final composition.

In other embodiments, preservative-free liquid formulations and compositions of the present invention can also be provided in single unit-dose containers. Such containers are acceptable to deliver the therapeutic dose of the compositions of the invention, particularly via injection. In certain such embodiments of the invention, the compositions can be effectively contained in a package comprising a container with a volume capacity of about 1 ml to about 10 ml. In other such embodiments of the invention, particularly those in which the compositions of the invention are provided in a dosage form suitable for parenteral administration, e.g., via intra-articular injection, the compositions can be effectively contained in a package comprising a syringe containing one or more of the compositions of the invention, particularly wherein the syringe containing the composition is itself contained within sterile packaging; in such embodiments, the sterile packaging is opened, and the composition of the invention is delivered to the affected joint of the patient, e.g., via intra-articular injection, and the syringe and packaging are then discarded. This use of single unit-dose containers eliminates the concern of contamination for the user (or other outside sources), as once the unit-dose container is opened and a single dose of the present formulations or compositions is delivered, the container is discarded.

Method of Use

Also provided herein is a method of treating a joint condition in a subject in need thereof by administering the subject, via an intra-articular injection, an acidic composition that includes an effective amount of rhGDF-5. Also provided herein is the use of an acidic composition that includes an effective amount of rhGDF-5 for the treatment of a joint condition in a subject in need thereof. “Conditions” as used herein include diseases and disorders but also refer to physiologic states. Typically, a joint condition is osteoarthritis and/or symptoms associated therewith.

In practicing such methods, the compositions are administered to the subject via intra-articular injection into the afflicted joint or the surrounding articular space. Methods of intra-articular injection of pharmaceutical compositions are well within the level of skill of the ordinarily skilled artisan, and are also described herein below. In some embodiments, the formulations described herein can be used in the methods.

The compositions are used to prevent or slow down (lessen) an undesired physiological change or disorder, such as the development and/or progression of osteoarthritis. Beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. Symptoms may include pain, inflammation, stiffness, muscle weakness, swelling, deformed joints, and/or erosion of cartilage. Efficacy of the formulation can be evaluated by any methods known in the field. In one exemplary embodiment, OA structural progression can be measured by joint space narrowing (JSN) on plain X-rays.

In some embodiments, the compositions and formulations described herein can stimulate cartilage regeneration. For example, after treatment, cartilage regeneration would be increased by about 5% or greater (10%, 20%, 30%, 40%, 50%, 60%, 70%, 80% or greater) relative to the level prior to treatment.

In some embodiments, the compositions and formulations described herein can reduce the width of at least one bone lesion. For example, after treatment, the width of at least one bone lesion would be reduced by about 5% or greater (10%, 20%, 30%, 40%, 50%, 60%, 70%, 80% or greater) relative to the width prior to treatment.

Suitable dosages (e.g., amounts, volumes, etc.) of the compositions of the invention may vary. One skilled in the art will appreciate that it is sometimes necessary to make routine variations to the dosage depending on the age and condition of the subject.

The effective amount of rhGDF-5 present in the acidic composition can vary. In some embodiments, the effective amount of rhGDF-5 is about 1.2 mg to about 30 mg in the acidic composition per injection. For example, the effective amount of rhGDF-5 is about 1.2 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg or 30 mg in the acidic composition. In some embodiments, the effective amount of rhGDF-5 is about 1.8 mg to about 6 mg in the acidic composition per injection.

In other embodiments, the effective amount of rhGDF-5 can stimulate cartilage regeneration by 5% or greater (10%, 20%, 30%, 40%, 50%, 60%, 70%, 80% or greater) relative to the level in the control groups. In other embodiments, the effective amount of rhGDF-5 can reduce the width of at least one bone lesion by 5% or greater (10%, 20%, 30%, 40%, 50%, 60%, 70%, 80% or greater) relative to the width in the control groups.

The concentration of rhGDF-5 present in the acidic composition can also vary, but in an exemplary embodiment rhGDF-5 is provided at a therapeutically effective amount. In some embodiments, the final concentration of rhGDF-5 in the acidic composition is about 0.6 mg/ml to about 3.0 mg/ml. For example, the final concentration of rhGDF-5 in the acidic composition is about 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3 mg/ml or any range derivable therein. Lower concentrations may be used if more frequent injections are used.

In some embodiments, a per injection volume of between about 2 mL to about 10 mL (suitably about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL or about 10 mL) of one or more of the compositions described herein is introduced into the subject.

In some embodiments, the acidic composition has a pH value of about 2.5 to about 5.0. For example, the acidic composition has a pH value of about 2.5 to about 4.0, about 2.5 to about 3.5 or about 2.5 to 3.0. For example, the formulation has a pH value of about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5. Typically, the acidic composition has a pH value of about 3.0. Typically, the acidic composition is glycine buffered.

In some embodiments, the acidic composition contains trehalose, sucrose, raffinose, glucose, mannitol or any combination thereof. Typically, the acidic composition contains trehalose. In some embodiments, the acidic composition contains glycine buffered trehalose.

In some embodiments. the trehalose is about 0.1% to about 50% by weight of the acidic composition. For example, the trahalose is about 0.10%, 0.50%, 0.10%, 0.50%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50% or 50.00% by weight of the acidic composition. Typically, the trahalose is about 5.0% by weight of the acidic composition.

As used herein, a “subject in need thereof” is a subject having a joint condition or a subject having an increased risk of developing a joint condition relative to the population at large. Risk factors for the development and/or progression of OA include, but are not limited to, local biomechanical factors like obesity, joint injury, joint deformity, and extensive sport participation, as well as systemic factors including age, gender, ethnic characteristics, bone density, and estrogen deficiency.

In some embodiments, the joint condition is osteoarthritis or symptoms associated therewith. Any joint can be treated by the compositions/formulations described herein, for example, knees, hands, feet, hips, elbow or shoulder.

Examples of symptoms or conditions, for which the composition and methods disclosed herein can be useful, encompass treating articular disorders, such as arthritis caused by infections, injuries, allergies, metabolic disorders, etc., rheumatoids such as chronic rheumatoid arthritis, and systemic lupus erythematosus; articular disorders accompanied by gout, arthropathy such as osteoarthritis, internal derangement, hydrarthrosis, stiff neck, lumbago, etc. Varying the effects depending on the use of the composition or the types of diseases to be treated, the agent can exert desired prophylactic and alleviative effects, or even therapeutic effects on swelling, pain, inflammation, and destroying of articulations without seriously affecting living bodies. The composition for treating articular disorder can be used to prevent the onset of articulation disorders, as well as to improve, alleviate, and cure the symptoms after their onsets.

According to the methods of the invention, the compositions described herein can be administered to the patient according to a wide variety of dosing schedules. For example, the compositions can be administered via intra-articular injection once in a treatment period. Alternatively, the composition can be administered via intra-articular injection once weekly for a treatment period (e.g., three weeks). A treatment period can be a predetermined amount of time (e.g., three to eight weeks (2, 3, 4, 5, 6, 7 or 8 weeks), or more, such as 2-6 months or up to one year (2, 3, 4, 5, 6, 7, 8, 9, 10, 11 or 12 months)). In some embodiments, treatment would range from: once weekly for three weeks, out to a single injection every 2-6 months.

A specific example of a “once weekly” dosing schedule is administration of the compositions of the invention on days 1, 8, 15 and 22 of the treatment period. In alternative embodiments, the compositions of the invention may be administered intermittently over a period of months. For example, the compositions of the invention may be administered weekly for three consecutive weeks biannually (i.e., repeat the weekly dosing schedule every six months), or they may be administered once a month for a period of two, three, four, five, six, seven, eight or more months. It will be appreciated that such administration regimens may be continued for extended periods (e.g., on the order of years) to maintain beneficial therapeutic effects provided by initial treatments. In yet other embodiments, such maintenance therapy may be effected following an acute dosing regimen designed to reduce the immediate symptoms of the joint condition, such as osteoarthritis. In most embodiments, however, the compositions of the invention are administered to the patient according to the methods described herein at least until the symptoms of the joint condition, such as OA, are alleviated or reduced. More commonly, the compositions of the invention and methods of the invention are used for a period of time after the symptoms are reduced to a tolerable level or completely eliminated so as to result in an improvement in the physiological structure of the joint by reducing or eliminating the underlying physiological causes of the joint condition.

The amount of active component(s) of the compositions (e.g., rhGDF-5) administered each time throughout the treatment period can be the same; alternatively, the amount administered each time during the treatment period can vary (e.g., the amount administered at a given time can be more or less than the amount administered previously). For example, doses given during maintenance therapy may be lower than those administered during the acute phase of treatment. Appropriate dosing schedules depending on the specific circumstances will be apparent to persons of ordinary skill in the art. For example, the dose of rhGDF-5 present in the composition may vary in the range of about 0.6 mg/ml to about 3.0 mg/ml per injection. For example, the dose of rhGDF-5 in the composition, per injection, is about 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3 mg/ml or any range derivable therein.

It will be understood, however, that the specific dosage level for any particular patient will depend upon a variety of factors including the activity of the specific compound employed, the age, body weight, general health, sex, diet, time of administration, rate of excretion, drug combination and the severity of the particular disease undergoing therapy. The pharmaceutical compositions can be prepared and administered in dose units. Under certain circumstances, however, higher or lower dose units may be appropriate. The administration of the dose unit can be carried out both by single administration of the composition or administration can be performed in several smaller dose units and also by multiple administrations of subdivided doses at specific intervals.

It will be readily apparent to one skilled in the relevant art that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

In one embodiment, the medical condition is osteoarthritis (OA) and the composition is administered in a joint space, such as, for example, a knee, shoulder, temporo-mandibular and carpo-metacarpal joints, elbow, hip, wrist, ankle, and lumbar zygapophysial (facet) joints in the spine. The viscosupplementation may be accomplished via a single injection or multiple intra-articular injections administered over a period of weeks into the knee or other afflicted joints. For example, a human subject with knee OA may receive one, two, three, or more injections or number of injections according to any suitable administration schedule of about 0.5, 1, 1.5, 2, 3, 4, 5, 6, 7, 8, 9, 10 ml or more per knee during the treatment period. For other joints, the administered volume can be adjusted based on the size on the joint, and the subject may receive one, two, three, or more injections or number of injections according to any suitable administration schedule during the treatment period.

A person skilled in the art will appreciate that the compositions and methods described herein can include various other joint treatment components (combination therapy), including, for example, amino acids, proteins, saccharides, di-saccharides, poly-saccharides, lipids, nucleic acids, buffers, surfactants, and mixtures thereof. Other useful components can include steroids, anti-inflammatory agents, non-steroidal anti-inflammatory agents, analgesics, cells, antibiotics, antimicrobial agents, anti-inflammatory agents, growth factors, growth factor fragments, small-molecule wound healing stimulants, hormones, cytokines, peptides, antibodies, enzymes, isolated cells, platelets, immunosuppressants, nucleic acids, cell types, viruses, virus particles, essential nutrients or vitamins, and combinations thereof. “Combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. The therapeutic agents can be administered by the same route or by different routes. For example, a first therapeutic agent of the combination selected may be administered by intra-articular injection while the other therapeutic agents of the combination may be administered orally. Alternatively, for example, all therapeutic agents may be administered by intra-articular injection. The sequence in which the therapeutic agents are administered is not narrowly critical. Therapeutic agents may also be administered in alternation.

Kits

The methods and compositions encompass kits for treating articular disorders, such as joints. The kits can include a solution that comprises an acidic vehicle and an effective amount of rhGDF-5 and optionally at least one additional component (such as stabilizers, excipients, pharmaceutically acceptable carriers and other treatment components). The solution has a pH value of about 2.5 to about 5.0. Typically, the solution has a pH value of about 3.0. The effective amount of rhGDF-5 is about 1.2 mg to about 30 mg. The acidic vehicle contains trehalose, or glycine buffered trehalose.

The solution including an acidic vehicle and an effective amount of rhGDF-5 can be housed in a separate container as the at least one additional component. In some embodiments, the solution including an acidic vehicle and an effective amount of rhGDF-5 can be housed in a separate chamber of a syringe as the at least one additional component. In other embodiments, all the components can be housed in a single chamber of a syringe for injecting.

The kits also include at least one syringe for injection. Examples can be for the knee, shoulder and hip, the volume of injection for a single injection product can be in the range of about 2 ml to 10 ml and the hand can be in the range of about 500 μl and 1.5 ml. The at least one additional component can be lyophilized or, alternatively can be in a solution.

The at least one additional component can be stored separately to increase shelf-life. The individual additional component can be lyophilized or in solid form in one syringe/cartridge with diluent or a second compound in a second syringe/cartridge. In one embodiment, one of the additional components is in lyophilized form or in solid form and the second component is a solution capable of combining with the lyophilized/solid component. An example can be at least one lyophilized or solid additional component can be stored in a first chamber and a solution that contains a second additional component can be stored in a second chamber.

Pre-filled dual-chamber syringes and/or cartridges can also be utilized with the additional components. Pre-filled dual-chamber syringes enable the sequential administration of two separate compositions with a single syringe push, thereby replacing two syringes with one. The benefits of a single delivery capability include increasing the speed and ease of drug administration; reducing risk of infection by reducing the number of connections; lowering the risk of drug administration or sequence errors, and quicker delivery of compositions requiring combination prior to administration. The dual-chamber syringe can accommodate lyophilized, powder or liquid formulations in the front chamber combined with diluents, saline or buffer in the rear chamber.

Prefilled syringes can contain the exact deliverable dose of desired compounds and diluents. The prefilled syringes can contain volumes from about 0.1m1, 0.2 ml, 0.3 ml, 0.4 ml, 0.5 ml, 0.6 ml, 0.7 ml, 0.8 ml, 0.9 ml, 1.0 ml, 1.5 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml or more or any derivative therein.

The dual syringe and/or cartridge can be side-by-side chambers with separate syringe plungers that mix into a single chamber or linear chambers with one plunger. The dual chamber syringe and/or cartridges can also have a stopper or connector in the middle to serve as a barrier between the two chambers. The stopper or connector can be removed to allow mixing or combining of the compounds in the two chambers.

A person skilled in the art will appreciate that any dual chamber systems known in the art can be used, and that the chambers can be side-by-side chambers with separate syringe plungers that mix into a single chamber or linear chambers with a single plunger.

Experimental Data

The data presented herein has demonstrated that Intra-articular injections of recombinant human growth differentiation factor-5 (rhGDF-5) containing formulations can successfully preserve and even regenerate cartilage in an animal model of osteoarthritis (OA). Although other bone growth factors demonstrated some cartilage preserving effects, they were observed to induce formation of ectopic bone and/or cartilage in the synovial tissue, while rhGDF-5 did not. Further, the use of an acidic vehicle (glycine buffered trehalose, pH=3) for rhGDF-5 did not produce detectable injection site pain or tissue damage. Moreover, the acidic vehicle alone produced an unexpected reduction in cartilage erosion.

A rat medial meniscus transection (MMT) model of OA which causes rapid and progressive OA-like pathology including the aggressive erosion of articular cartilage was used in these studies. Specifically, OA was surgically induced in male Lewis rats (200-225 g) by transection of the medial collateral ligament and medial meniscus of the femoro-tibial joint. Three studies were conducted, each using 15 rats per group to investigate the feasibility of rhGDF-5 in either a preventative or therapeutic modality.

In all studies, joints were harvested and processed for histological analysis following staining with toluidine blue. Quantitative and semi-quantitative histopathological end points were used to evaluate the extent of OA changes in the medial tibial plateau cartilage. Further, all animals were monitored daily for evidence of swelling/inflammation, as well as for excessive pain or behavioral changes resulting from the injections.

The rat MMT model is a well-accepted model of both pain and joint deterioration observed in human OA, and human OA therapies are known to be efficacious in this model. For example, Bove et al (Osteoarthritis and Cartilage, (2006) 14, 1041-1048) showed pain reduction in the rat MMT model using an oral dose of rofecoxib (a.k.a. Vioxx) of 10 mg/kg. Typical human dosages of rofecoxib are in the range of 0.3-0.7 mg/kg. The fact that human dosing is lower than rat dosing, on a body weight basis, is consistent with the commonly used practice of allometric scaling, which is used to extrapolate human dosing from animal dosing, based on established relationships between metabolism, body size, and weight in different species. It is common practice to scale up dosing from one species to another by using the ratio of animal weights to the ¾ power:

Dose 2=Dose 1 [(weight 2)**0.75/(weight 1)**0.75]

Typical rat and human weights are 0.3 and 70 kg, respectively. To extrapolate a dose of 100 microgram of rhGDF-5 in a rat to human dosing, allometric scaling would predict:

Dose 2=(100 μg)*[(70 kg)**0.75/(0.3 kg)**0.75]=5970 microgram=5.97 mg

It is common to use volumes ranging from 2-10 ml of solution for intra-articular injection in humans. Thus, it is reasonable to expect that single intra-articular injections comprising from 0.6-3 mg/ml rhGDF-5 would be efficacious in human subjects. Lower concentrations would be expected to show efficacy if more frequent injections were used. For example, if we extrapolated the 30 microgram dose for rats to humans, the human dose would be 1.8 mg, and the concentration would range from 0.18-0.9 mg/ml rhGDF-5.

### Example 1: The Effect of rhGDF-5 on Preventing Progression of OA

Six weekly injections began on day 3 post-MMT with termination on day 42 (preventative). Controls for this study were the glycine buffered trehalose vehicle (pH=3) and formulations of growth factors FGF-18 and BMP-7 (a.k.a. OP-1), both of which have been studied for enhancing bone and/or cartilage regrowth, and have been proposed for the treatment of OA via intra-articular injection.

In this study, rhGDF-5 demonstrated significant dose-dependent cartilage protection as a preventative therapy. In particular, as shown in FIG. 1, the width of significant tibial lesions was reduced by 11% by repeat treatment with 0.3 μg rhGDF-5, 22% by 3 μg rhGDF-5, 32% by 10 μg rhGDF-5, and 49% by 30 μg rhGDF-5 injections when compared with vehicle controls.

Although both the FGF-18 and BMP-7 groups also showed cartilage preservation, both compounds resulted in an increase in osteophyte formation compared to vehicle. As shown in FIG. 3, the FGF-18 group showed an approximately 2-fold increase in osteophyte size compared to vehicle. The high dose OP-1 group showed a 50% increase over vehicle. GDF-5 did not produce osteophyte enlargement compared to vehicle at any of the doses studied. Also, formation of ectopic bone was observed in the synovium of 7% of the animals in the low dose OP-1 group, and in all animals in the high dose OP-1 groups. This side effect was not observed with GDF-5. Additionally, significant thickening of the medial collateral ligament, relative to vehicle and to GDF-5, was observed with the high dose OP-1 group. Finally, significant synovial inflammation was observed for GDF-5, OP-1 and FGF-18. However, the extent of inflammation was significantly higher in the FGF-18 and high dose OP-1 groups, with all animals in these groups showing severe inflammation. All side effects are summarized in Table 1.

### Example 2: The Efficacy of rhGDF-5 as Preventative and Therapeutic Approach

In this study, 6 weekly or 3 bi-weekly injections began on day 7 post-MMT (preventative or “Pre”) or day 21 post-MMT (therapeutic or “Ther”) with termination on day 63. Control for this study was the trehalose vehicle.

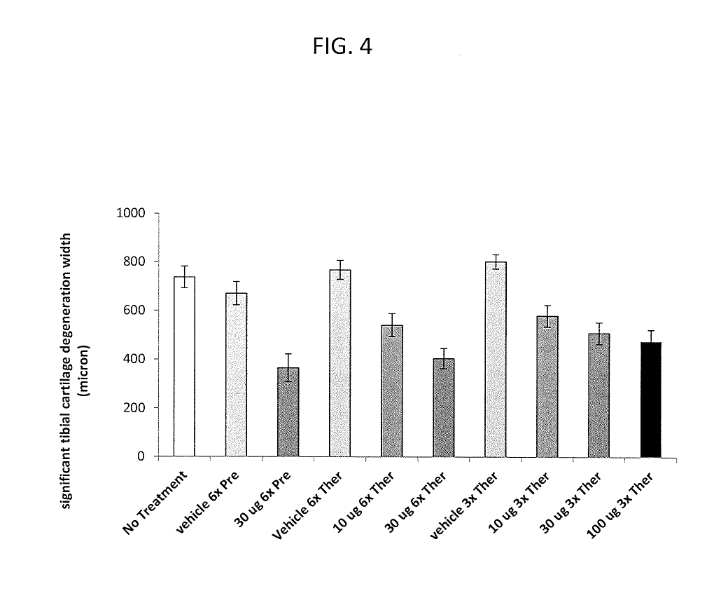

The results in this study demonstrated dose-dependent efficacy of rhGDF-5 compared with vehicle controls when administered in a therapeutic modality beginning on day 21 post-MMT. As shown in FIG. 4, following 6 weekly injections, the width of significant tibial lesions was reduced by 30% by a 10 μg rhGDF-5 dose and by 47% by a 30 μg dose. Following a 3 bi-weekly injection regimen there was a 28% and a 37% reduction in the width of significant tibial lesions, respectively, for 10 μg and 30 μg doses. This reduction in efficacy was partially compensated for by increasing the rhGDF-5 dose to 100 μg, which reduced lesion width by 41%. Even at the higher rhGDF-5 doses in this study, no formation of ectopic cartilage or bone was observed.

### Example 3: The Efficacy of rhGDF-5 as Therapeutic Approach

A single injection, or a series of 2 or 3 bi-weekly injections, began on day 21 post-MMT, with termination on day 63 (therapeutic). Controls for this study were the trehalose vehicles. Also included in this study was an untreated group that was sacrificed at day 21 post-MMT, so that comparisons could be made between cartilage erosion at day 21 and cartilage erosion in groups treated with rhGDF-5 beginning at day 21.

In this study, a single rhGDF-5 injection on day 21 reduced the width of significant tibial lesions compared with vehicle controls by 13% and 19% for a 30 μg and 100 μg dose respectively. The 2-injection regimen administered on days 21 and 35 post-MMT reduced tibial lesion width by 18% and 28%, respectively, and the 3-injection regimen reduced these lesions by 27% and 43% respectively. After accounting for a modest vehicle effect, the level of Day 63 cartilage degeneration observed with 3 biweekly injections of rhGDF-5 (30 or 100 μg) was significantly less than the untreated controls at day 21. This is indicative of disease arrest and cartilage regeneration with repeated injections of higher doses of rhGDF-5, again with no ectopic tissue formation. Results from 30 μg and 100 μg dosing of rhGDF-5 are shown in FIG. 5. Histological cross sections showing cartilage preservation, using the 100 μg dose, are shown in FIG. 6.

All of the rhGDF-5 formulations in the studies described above used a vehicle comprising glycine buffered trehalose (pH=3.0). None of the vehicle groups exhibited evidence of tissue damage resulting from the acidicity of the vehicle. In fact, the vehicle group in Example 1 showed an unexpected and significant reduction in cartilage lesion severity (see above). Improvement was also observed in the vehicle groups in Examples 2 and 3. Further, there was no evidence of pain or behavioral changes in the animals beyond the normally observed injection site pain.

The results demonstrated that intra-articular delivery of rhGDF-5 significantly protected the joint from MMT-induced osteoarthritis in a dose and administration regimen dependent manner, without the formation of ectopic bone or cartilage observed with other growth factors. Further, no negative effects, resulting from the use of an acidic vehicle for rhGDF-5 were observed. In contrast, unexpected benefits were observed from the use of such acidic vehicle for rhGDF-5.

The data also indicated that even a single injection of 100 μg rhGDF-5 has significant therapeutic benefit. Additionally, rhGDF-5 was able to arrest disease progression in a therapeutic modality with as few as 2 bi-weekly injections. Moreover, 3 bi-weekly injections of 100 μg rhGDF-5 were able to stimulate cartilage regeneration in the MMT model. Collectively, these results have clearly demonstrated the application of rhGDF-5 as a targeted therapeutic approach for treating OA.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An injectable formulation, comprising:
an effective amount of recombinant human growth differentiation factor-5 (rhGDF-5); and
an acidic vehicle, wherein the formulation is an intra-articular injection formulation.

2. The formulation of claim 1, wherein the acidic vehicle comprises trehalose, sucrose, raffinose, glucose, mannitol, sorbitol, erythritol or any combination thereof.

3. The formulation of claim 2, wherein the acidic vehicle comprises trehalose.

4. The formulation of claim 3, wherein the trehalose is about 0.1% to 50% by weight of the formulation.

5. The formulation of claim 3, wherein the trehalose is about 5% by weight of the formulation.

6. The formulation of claim 1, wherein the formulation has a pH of from about 2.5 to about 5.0.

7. The formulation of claim 1, wherein the formulation has a pH of about 3.

8. The formulation of claim 1, wherein the effective amount of rhGDF-5 is about 1.2 mg to about 30 mg.

9. The formulation of claim 1, wherein the formulation comprises a diluent selected from the group consisting of water, saline, and a buffer.

10. The formulation of claim 9, wherein the buffer is a glycine buffer.

11. A method of treating a joint condition in a subject in need thereof, comprising administering to the subject via an intra-articular injection an acidic composition comprising an effective amount of rhGDF-5.

12. The method of claim 11, wherein the method comprises a single injection in a treatment period.

13. The method of claim 11, wherein the method comprises injections once every week in a treatment period.

14. The method of claim 11, wherein the acidic composition comprises trehalose.

15. The method of claim 11, wherein the acidic composition comprises glycine buffered trehalose.

16. The method of claim 11, wherein the acidic composition has a pH of from about 2.5 to about 5.0.

17. The method of claim 11, wherein the acidic composition has a pH of about 3.

18. The method of claim 11, wherein the effective amount of rhGDF-5 is about 1.2 mg to about 30 mg.

19. The method of claim 11, wherein the joint condition is osteoarthritis.

20. The method of claim 11, wherein the effective amount of rhGDF-5 stimulates cartilage regeneration.

21. The method of claim 11, wherein the effective amount of rhGDF-5 reduces the width of at least one bone lesion.

22. A kit, comprising:
a container comprising a solution having a pH in the range of about 2.5 to about 5.0 comprising an acidic vehicle and an effective amount of rhGDF-5; and
a syringe for injection of the solution.

23. The kit of claim 22. wherein the effective amount of rhGDF-5 is about 1.2 mg to about 30 mg.

24. The kit of claim 22, wherein the acidic vehicle comprises trehalose.
